(19) United States
(12) Patent Application Publication
(10) Pub. No.: US 2007/0062063 A1
(43) Pub. Date: Mar. 22, 2007

(54) Mobile mattress sanitizer

(76) Inventor: Vernon W. Langley, Gamer, NC (US)

Correspondence Address: LITMAN LAW OFFICES, LTD, PO BOX 15035, CRYSTAL CITY STATION, ARLINGTON, VA, 22215, US

(21) Appl. No.: 11/497,247

(22) Filed: Aug. 2, 2006

Related U.S. Application Data
(60) Provisional application No. 60/714,369, filed on Sep. 7, 2005.

(51) Int. Cl. D06F 35/00; B08B 3/00; A61L 2/00; F26B 3/00; D06F 29/00

(52) U.S. Cl. 34/507; 68/18 R; 134/115 R; 422/24

(57) ABSTRACT

The mobile mattress sanitizer is a mobile unit, such as a truck, van, trailer or the like, having a mobile cargo space that includes a heater and heater fan mounted in an upper region of the cargo space. Heating fuel is supplied by an external fuel tank. An operator has access to a thermometer, thermostat and timer for precise sterilization control. A power switch is also mounted for easy access to a sanitizer operator. A wheeled rack having storage space for multiple mattresses is provided. A power lift gate- is included for lifting the mattress rack onto the unit. Guide tracks guide a plurality of racks along a predetermined path within the unit. When the racks with mattresses are in place, a door seals the cargo space and the operator initiates thermal warming of air within the environment to a predetermined temperature for a predetermined time for mattress sanitization.

### CROSS-REFERENCE TO RELATED APPLICATION

This application claims the benefit of U.S. Provisional Patent Application Serial No. 60/714,369, filed Sep. 7, 2005.

### BACKGROUND OF THE INVENTION

1. Field of the Invention

The present invention relates to devices for sterilizing bedding materials and furnishings, and particularly to a mobile mattress sanitizer for killing dust mites and other microorganisms deep inside a mattress, box spring, cushion, upholstery and the like.

2. Description of the Related Art

Bedding material is often a breeding ground for dust mites and other insects that carry microorganisms that can be harmful to one's health. Such infestations may have their source in the manufacturing facility, or in warehouses or retail stores pending sale. Such infestations may also occur after sale, and ay be a particular problem in hospitals, hotels, motels, nursing homes, dormitories, and other institutions that accommodate a transient population. Cushions used on couches and chairs may have similar problems.

Chemical sprays or fumigants are often effective in killing pests or microorganisms on the surface of the mattress, but are usually not effective in killing pests and microorganisms deep within the mattress. Consequently, the same problems tend to recur days or weeks later, and the treatment needs to be repeated. Heat is an effective treatment for killing such pests, but sending individual mattresses and box springs out for a thorough course of treatment is not very economical, considering the cost of transportation and the probable loss of use of the bedding material for several days.

Thus, a mobile mattress sanitizer solving the aforementioned problems is desired.

### SUMMARY OF THE INVENTION

The mobile mattress sanitizer is a mobile unit, such as a truck, van, trailer, or the like, having a mobile cargo space that includes a heater and heater fan mounted in an upper region of the mobile cargo space. An exhaust vent and fan is located in a lower region of the mobile cargo space. A fuel source for the heater is mounted on the exterior of the mobile cargo space. A thermostat and timer are mounted for easy access and control of sterilization temperature and duration. A power switch is also mounted for easy access to an operator of the sanitizer. A thermostat is also provided so that the operator can easily monitor the temperature inside the cargo space. A wheeled rack having storage space for multiple mattresses permits sanitizing several mattresses at one time. A power lift gate is included for lifting the mattress rack onto the mobile cargo space. Guide tracks are provided for guiding a plurality of racks along a predetermined path within the mobile cargo space. When the racks with mattresses are in place, a door seals the cargo space environment so that the operator can initiate thermal warming of air within the environment to a predetermined temperature and for a predetermined time for sanitization of the mattresses.

These and other features of the present invention will become readily apparent upon further review of the following specification and drawings.

### BRIEF DESCRIPTION OF THE DRAWINGS

FIG. 1 is an environmental perspective view of a mobile mattress sanitizer according to the present invention, broken away and partially in section to show details of the housing and heating unit.

FIG. 2 is a cutaway environmental side view of the mobile mattress sanitizer according to the present invention.

FIG. 3 is a perspective view of a mattress rack used in the mobile mattress sanitizer according to the present invention.

FIG. 4 is a rear cutaway perspective view of the mobile mattress sanitizer according to the present invention, broken away and partially in section.

Similar reference characters denote corresponding features consistently throughout the attached drawings.

### DETAILED DESCRIPTION OF THE PREFERRED EMBODIMENT

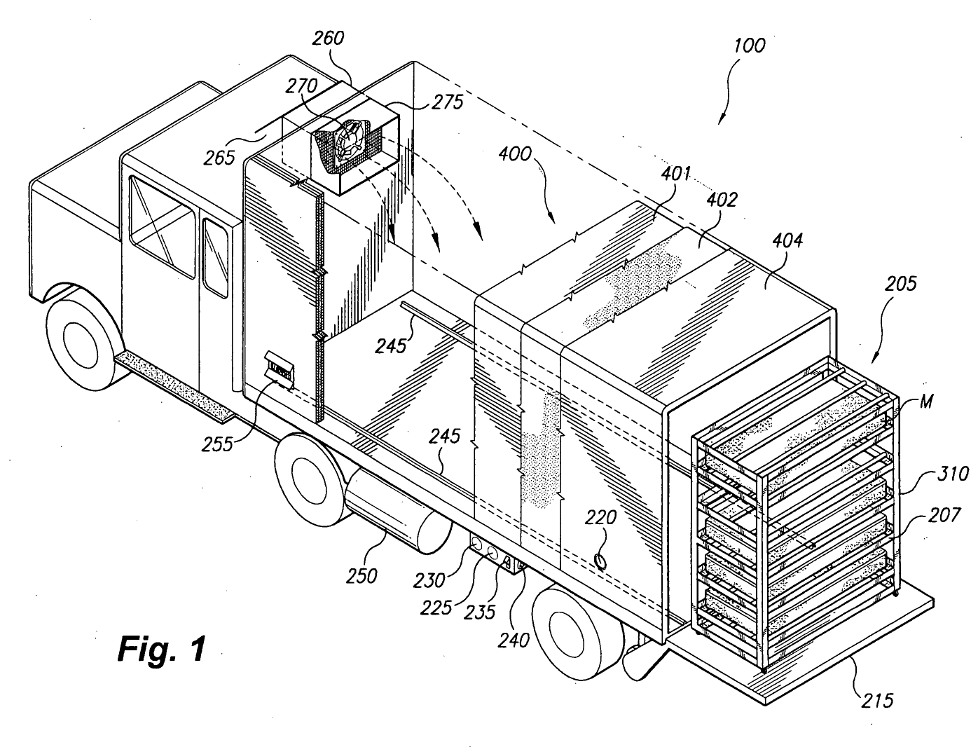

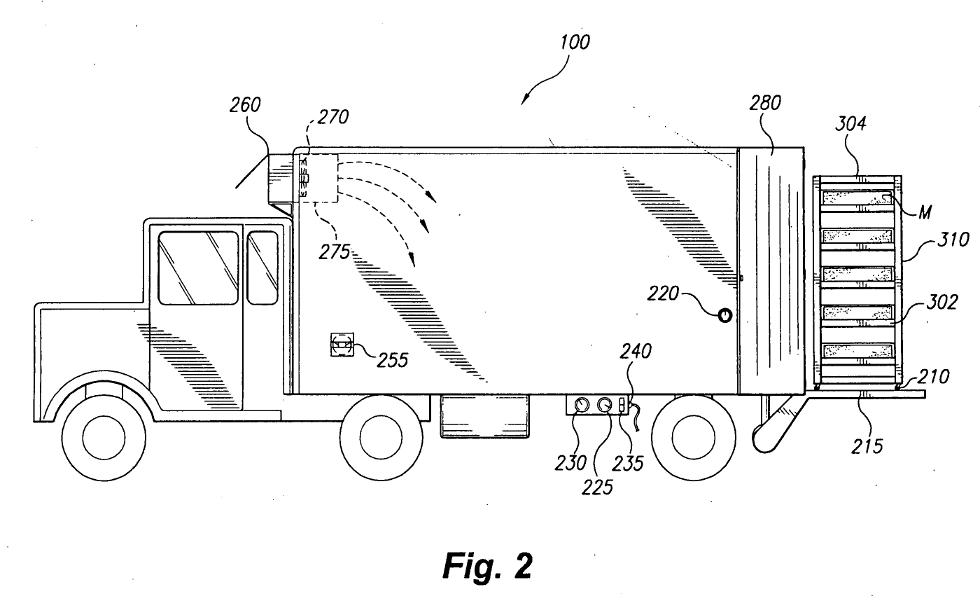

As shown in FIGS. 1, 2 and 4, the mobile mattress sanitizer 100 is a mobile unit, such as a truck, van, trailer, or the like, having a mobile cargo space 400 that includes a heating unit heating air in the cargo space 400 and forcing air into the cargo space 400 and an exhaust unit for removing hot air from the cargo space 400 in order to maintain a continuous airflow of heat for disinfecting the contents of the cargo space 400. As shown in FIG. 2, heating fuel is supplied by an external fuel tank, such as heater fuel tank 250, e.g., 50-gallon propane tank. The operator has access to a thermometer 220, thermostat 225 and timer 230 for control of the sanitizer 100.

An electric receptacle 240 providing electricity from a generator or other portable electrical power source is included to provide power to the heating and control system of the mobile mattress sanitizer 100. Alternatively, the receptacle 240 may be adapted to receive an extension cord from an ac power main for distributing power from an external power supply to the heating, control, and exhaust components. Voltage being received by the receptacle 240 may be, e.g., 120 volts, 240 volts, and the like. A power switch 235 for switching power to heater control circuitry and electric fans is also mounted for easy access to the operator of the sanitizer 100.

As shown in FIGS. 1-4, a wheeled rack 205 having storage space for multiple mattresses is provided. A power lift gate 215 is included for lifting the mattress rack 205 into the cargo space 400 of the sanitizer 100. Guide tracks 245 guide a plurality of racks along a predetermined path within the mobile cargo space 400. When the racks 205 have been loaded with mattresses M and wheeled into position utilizing guide tracks 245, a door 280 sealing off the mobile cargo space 400 is closed.

Utilizing the heater thermostat 225 and the electric timer 230, the operator initiates thermal warming of air within the environment to provide heated air at a predetermined temperature and for a predetermined time for complete mattress and/or box spring sanitization, resulting in killing dust mites and other microorganisms deep inside the mattress and/or box spring.

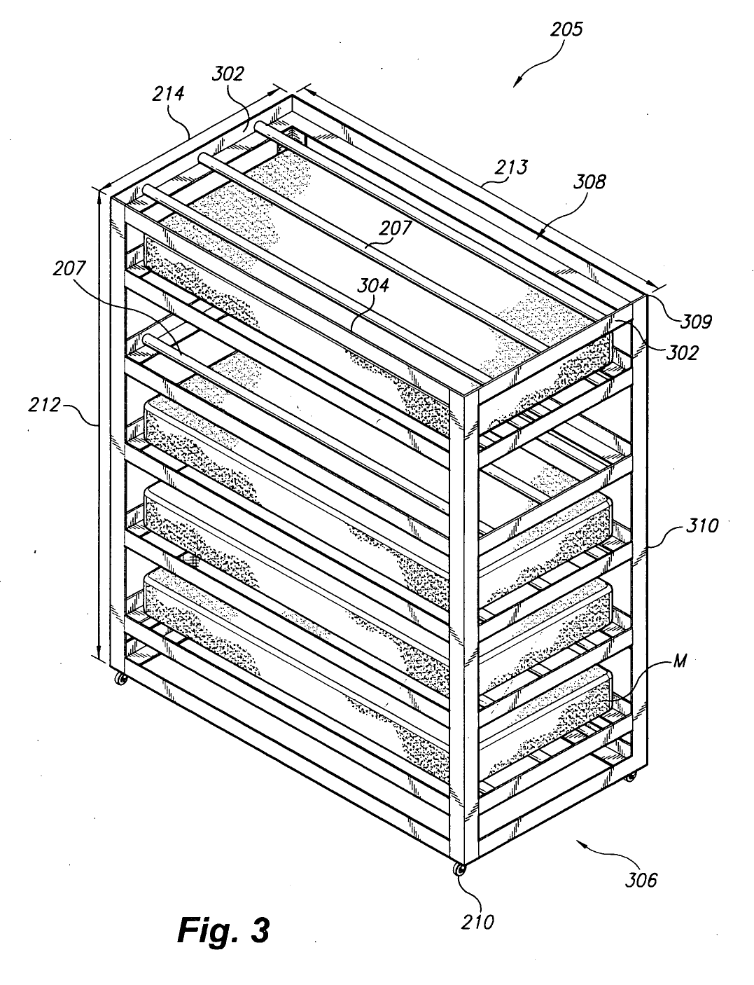

Referring to FIG. 3, the rack 205 is a four-sided open structure that includes a plurality of wheels 210. The rack height 212 should be sufficient for storage of a plurality of mattresses M while also maintaining an air gap between each of the mattresses. An exemplary rack height 212 is approximately five feet, eight inches high. Rack width 214 should be wide enough to accommodate up to a king-size mattress; therefore, preferably a rack width 214 of greater than 76″ is provided. Correspondingly, rack length 213 is preferably greater than 80″ to accommodate a king-size mattress.

Support beams 304 are configured to form a rectangular base 306, a rectangular top 308, and four posts 310 are disposed between the base 306 and the top 308 to form the corners 309 of the rack 205. The structure may be welded, bolted, or otherwise fastened together.

Support beams 304 may be steel or aluminum angles to facilitate ease of construction of the mattress rack 205. Similarly, lateral members 302 extend between posts 310 to form the frame for each shelf. Mattress support rods 207 are disposed in a parallel fashion between, and fastened to, the lateral members 302. Spacing of the mattress support rods 207 should be provided to maintain sufficient support of the mattress M while maintaining sufficient airflow for efficient sanitization. For this purpose, mattress support rods 207 may be spaced approximately 14″ apart. Several shelves or levels of mattress support rods 207 are provided in a like manner with sufficient vertical separation between levels to allow an air gap, preferably greater than 5″, to be maintained between each of the mattresses M loaded on to the mattress rack 205.

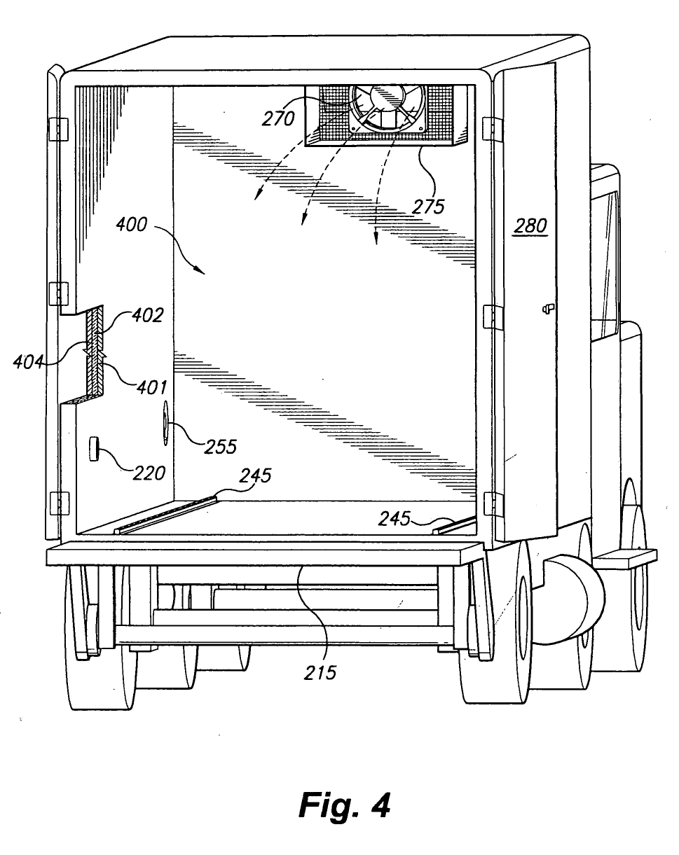

As shown in FIG. 4, guide tracks 245 are raised metal rails provided to restrict lateral movement of the rack 205. Utilization of the guide tracks 245 permits an operator to efficiently load and unload racks 205 without struggling to keep them in an orderly position in the mobile cargo space 400.

The walls of the mobile cargo space 400 form the housing of the sanitizer 100 and are provided with thermal insulation 402. As shown in FIGS. 1 and 4, mobile cargo space 400 has an outer wall 404, an inner wall 401, and sandwiched in between is the thermal insulation 402. Thermal insulation 402 may comprise, e.g., 2″ of duct insulation. Insulation 402 increases the efficiency of the heating process provided by heater 260.

As stated above, heater thermostat 225 and electric timer 230 provide heater control inputs for the operator to dial in a predetermined temperature and duration of mattress sanitization. Preferably, the predetermined temperature for effective mattress sanitization is at least 240° F., which has been determined to provide effective neutralization of dust mites and other pests, microorganisms, and their waste products. Heating duration for effective sanitization deep inside the mattress M preferably ranges from about forty-five minutes to approximately two hours, depending upon the cargo load.

Controls may be preset so that the system never exceeds a predetermined safe temperature limit. According to the present invention, a preferable predetermined safe temperature limit is approximately 251° F. Thermometer 220 is provided for the operator to visually monitor the ambient temperature of the mobile cargo space 400.

The heater 260 has sufficient thermal heating capacity so that a mobile cargo space 400 having the dimensions of an average 24-foot cargo truck can achieve and maintain the desired temperature range for the above time intervals. A 375,000 BTU heater unit is adequate in most circumstances. Placement of the heater 260 should be in an upper region of the mobile cargo space 400, and should provide a clear, unimpeded flow of fresh air into heater intake 265. Enclosing the heater blower fan 270 is a rectangular shaped conduit 275 with an opening to the rear of the heater blower fan 270 in order to provide a directed flow of freshly heated air that circulates to the rear of the mobile cargo space 400 and over the mattresses M. A squirrel cage circulation fan (not shown) may also be added to assist in circulating the heated air within the mobile cargo space 400. An exhaust vent with fan 255 is preferably located in a bottom region of the mobile cargo space 400. Thus, a continuous airflow of freshly-heated air is introduced near the top of mobile cargo space 400, while at the same time continuously exhausting heated air near the bottom of mobile cargo space 400. Additionally, a data recorder (not shown) that records the cargo space temperature vs. time during operations of the mobile mattress sanitizer 100 may be provided.

With modification to the mattress rack 205, other types of furnishing and items, such as cushions for couches, chairs, and the like, may be supported by the rack 205 for sanitization by the sanitizer 100. In addition, more heat circulation could be provided by the addition of fans throughout the mobile cargo space 400.

It is to be understood that the present invention is not limited to the embodiment described above, but encompasses any and all embodiments within the scope of the following claims.

I claim:

1. A mobile mattress sanitizer, comprising:
a vehicle having a cargo space;
at least one rack having a plurality of shelves, each of the shelves being adapted for receiving a mattress, the rack being received within the cargo space; and
a heater for heating the cargo space to a temperature sufficient to kill harmful pests and microorganisms in the mattresses;
whereby a plurality of mattresses are sanitized simultaneously.

2. The mobile mattress sanitizer according to claim 1, further comprising: a heater fan in addition to the heater; the heater fan being provided for forcing heated air into the cargo space; both heater and heater fan being mounted in an upper region of the mobile cargo space.

3. The mobile mattress sanitizer according to claim 1, further comprising: the heater having sufficient thermal heating capacity to heat an average 24-foot cargo truck cargo space to approximately 240° F. for a minimum time period of approximately 45 minutes.

4. The mobile mattress sanitizer according to claim 3, further comprising: a heater thermostat; an electric timer; and, wherein the heater thermostat and electric timer provide heater control inputs for an operator to dial in a predetermined temperature and duration of mattress sanitization.

5. The mobile mattress sanitizer according to claim 4, wherein the heater control inputs may have a preset so that a predetermined safe temperature limit is never exceeded.

6. The mobile mattress sanitizer according to claim 5, wherein the predetermined safe temperature limit is approximately 251° F.

7. The mobile mattress sanitizer according to claim 1, further comprising: a thermometer; the thermometer being capable of providing an operator with a visual indication of an ambient temperature of the cargo space.

8. The mobile mattress sanitizer according to claim 2, further comprising: a conduit that encloses the heater fan; the conduit having an opening to the rear of the heater fan in order to provide a directed flow of freshly heated air that circulates to the rear of the cargo space and over mattresses disposed in the cargo space.

9. The mobile mattress sanitizer according to claim 1, further comprising: an exhaust vent having a fan; the exhaust vent and fan being disposed in a bottom region of the cargo space.

10. The mobile mattress sanitizer according to claim 2, wherein a continuous airflow of freshly-heated air is introduced near the top of the cargo space, while at the same time heated air is continuously exhausted near the bottom of the cargo space.

11. The mobile mattress sanitizer according to claim 1, wherein the rack has wheels.

12. The mobile mattress sanitizer according to claim 1, wherein the vehicle has a power lift gate, the power lift gate being provided for lifting the rack into the cargo space.

13. The mobile mattress sanitizer according to claim 11, wherein guide tracks are disposed within the cargo space to guide a plurality of the racks along a predetermined path within the cargo space.

14. The mobile mattress sanitizer according to claim 1, further comprising: a door on the vehicle; the door being capable of sealing off the cargo space when closed.

15. The mobile mattress sanitizer according to claim 1, wherein dimensions of the rack are sufficient for storage of a plurality of mattresses while also maintaining an air gap between each of the mattresses.

16. The mobile mattress sanitizer according to claim 15, wherein dimensions of the rack are sufficient to accommodate up to a king-size mattress.

17. The mobile mattress sanitizer according to claim 1, further comprising: mattress support members being disposed on the rack in a manner that provides the capability to maintain sufficient support of a plurality of mattresses, the support members being spaced apart from each other to maintain sufficient airflow around the mattresses.

18. The mobile mattress sanitizer according to claim 1, wherein walls housing the cargo space have thermal insulation.

19. The mobile mattress sanitizer according to claim 18, wherein the walls comprise; an outer wall; an inner wall; the thermal insulation being disposed between the outer wall and the inner wall.

20. The mobile mattress sanitizer according to claim 1, wherein an electric receptacle is disposed on the vehicle to receive electricity from an electric power source in order to provide heating and control system power.
